(19) United States
(12) Patent Application Publication
(10) Pub. No.: US 2025/0354952 A1
(43) Pub. Date: Nov. 20, 2025

(54) ELECTRODE AND METHOD FOR MANUFACTURING ELECTRODE

(71) Applicant: Murata Manufacturing Co., Ltd., Nagaokakyo-shi (JP)

(72) Inventors: Kosuke SUGIURA, Nagaokakyo-shi (JP); Mayu HIRAYAMA, Nagaokakyo-shi (JP)

(21) Appl. No.: 19/283,288

(22) Filed: Jul. 29, 2025

Related U.S. Application Data
(63) Continuation of application No. PCT/JP2024/003081, filed on Jan. 31, 2024.

(30) Foreign Application Priority Data
Feb. 3, 2023 (JP) 2023-015495

(51) Int. Cl. G01N 27/327

(52) U.S. Cl. CPC G01N 27/327

(57) ABSTRACT

An electrode including: a film containing metal cation-containing layered material particles, the metal cation-containing layered material particles each have one layer or a plurality of layers and a metal cation, the one layer or each of the plurality of layers includes a layer body represented by: MₘXₙ, wherein M is at least one metal of Group 3-7 and includes at least a Ti atom, X is a carbon atom, a nitrogen atom, or a combination thereof, n is 1 to 4, m is more than n but not more than 5, a modifier or terminal T existing on a surface of the layer body, and a content of the metal cation is 0.004 mol or more per gram of the film; and a conductive gel portion in contact with the film.

### CROSS REFERENCE TO RELATED APPLICATIONS

The present application is a continuation of International application No. PCT/JP2024/003081, filed Jan. 31, 2024, which claims priority to Japanese Patent Application No. 2023-015495, filed Feb. 3, 2023, the entire contents of each of which are incorporated herein by reference.

### TECHNICAL FIELD

The present disclosure relates to an electrode and a method for manufacturing an electrode.

### BACKGROUND ART

In recent years, MXene has been attracting attention as a new material. MXene is a type of so-called two-dimensional material, and as will be described later, is a layered material in the form of one layer or a plurality of layers. In general, MXene is in the form of particles of such a layered material (which are called “layered material particles” and can include powders, flakes, nanosheets, and the like).

Currently, various studies are being conducted toward the application of MXene to various fields. For example, the application of MXene for use in which a high conductivity is required to be maintained, such as an electrode or an electromagnetic shield (EMI shield) in an electric device is under research. For example, Non-Patent Document 1 describes that Ti₃C₂ MXene, which is a two-dimensional material, is a material clearly different from carbon-based nanomaterials, and a Ti₃C₂ MXene microelectrode exhibits a superior low impedance as compared with existing metal microelectrodes, and is suitable for recording neural signals from a living body, for example, a brain. In addition, Non-Patent Document 2 also shows that MXene can be effective in many applications in the field of living bodies ranging, for example, from mapping of a wide range of human neuromuscular networks to cortical microstimulation in a small animal model.

Non-patent Document 1: Driscoll, Nicolette, et al. “Two-dimensional Ti3C2 MXene for high-resolution neural interfaces” ACS nano 12.10 (2018): 10419-10429

Non-patent Document 2: Driscoll, Nicolette, et al. “MXene-infused bioelectronic interfaces for multiscale electrophysiology and stimulation” SCIENCE TRANSLATIONAL MEDICINE (2021)

### SUMMARY OF THE DISCLOSURE

For example, to perform high-resolution sensing in the biological field, it is important to reduce the interface impedance of an electrode as much as possible. Some of the electrodes are provided with a conductive gel as a conductive film. However, in the case of an electrode provided with a conductive gel, it is difficult to reduce the impedance of the electrode, and it is considered that electrodes containing the MXenes described in Non-Patent Documents 1 and 2 are required to be improved for the purpose of reduction in impedance. The present disclosure has been made in view of the above circumstances, and an object of the present disclosure is to provide a low impedance electrode provided with a conductive gel and a method for manufacturing the same.

According to one aspect of the present disclosure, there is provided an electrode comprising: a film containing metal cation-containing layered material particles, wherein the metal cation-containing layered material particles each have one layer or a plurality of layers and a metal cation, the one layer or each of the plurality of layers includes a layer body represented by:

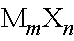

wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein Tis at least one selected from a hydroxy group, a fluorine atom, a chlorine atom, an oxygen atom, and a hydrogen atom, and
a content of the metal cation is 0.004 mol or more per gram of the film; and
a conductive gel portion in contact with the film.

According to another aspect of the present disclosure, there is provided a method for manufacturing an electrode, the method comprising:
(a) preparing layered material particles each including one layer or a plurality of layers, the one layer or each of the plurality of layers including a layer body represented by:

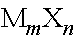

wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein T is at least one selected from a hydroxy group, a fluorine atom, a chlorine atom, an oxygen atom, and a hydrogen atom;
(b) mixing a dispersion liquid containing the layered material particles with an aqueous solution containing a metal cation to obtain a dispersion liquid containing metal cation-containing layered material particles;
(c) obtaining a film containing the metal cation-containing layered material particles, in which a content of the metal cation is 0.004 mol or more per gram of the film, using a dispersion liquid containing the metal cation-containing layered material particles; and
(d) forming a conductive gel portion on at least one surface of the film.

According to the present disclosure, there is provided an electrode including a conductive gel, in which a film contained in the electrode and containing particles of a prescribed layered material (also referred herein to as “MXene”) contains a certain amount or more of metal cations, and thus the electrode contains MXene and exhibits a low impedance. In addition, a manufacturing method by which the electrode can be easily manufactured is provided.

### BRIEF DESCRIPTION OF THE DRAWINGS

FIGS. 1(a) and 1(b) are schematic sectional views of MXene constituting the film included in the electrode of the present embodiment.

### DETAILED DESCRIPTION

### Embodiment 1: Electrode

Hereinafter, an electrode in one embodiment of the present disclosure will be described in detail, but the present disclosure is not limited to such an embodiment.

The electrode in the present embodiment is an electrode comprising a film containing metal cation-containing layered material particles and a conductive gel portion in contact with the film.

The metal cation-containing layered material particles each have one layer or a plurality of layers and a metal cation, and the one layer or each of the plurality of layers includes a layer body represented by the following formula:

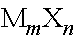

wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein T is at least one selected from the group consisting of a hydroxy group, a fluorine atom, a chlorine atom, an oxygen atom, and a hydrogen atom.

A content of the metal cation is 0.004 mol or more per gram of the film. As a result, it is possible to realize an electrode provided with a conductive gel, the electrode containing MXene and exhibiting a low impedance.

Hereinafter, a film that constitutes the electrode of the present embodiment and contains layered material particles each of which includes one layer or a plurality of layers and includes a metal cation (metal cation-containing layered material particles) (the film may be referred to as a “metal cation-containing MXene film” or a “conductive film”) will be described. The layered material can be understood as a layered compound and is also represented by “MₘXₙTₛ”, wherein s is any number and traditionally x or z may be used instead of s. Typically, n can be 1, 2, 3, or 4, but is not limited thereto.

In the above formula of MXene, M may be only Ti, or may have Ti and further have at least one selected from the group consisting of Zr, Hf, V, Nb, Ta, Cr, Mo, and Mn. When M includes an element other than Ti, the element other than Ti is more preferably at least one selected from the group consisting of V, Cr, and Mo.

Examples of MXene include those with the above formula MₘXₙ expressed as below.
Ti₂C, Ti₂N, (Ti, V)₂C, (Ti, Nb)₂C,
Ti₃C₂, Ti₃N₂, Ti₃ (CN), (Ti, V)₃C₂, (Ti₂Nb)C₂, (Ti₂Ta)C₂, (Ti₂Mn)C₂, (V₂Ti) C₂, (Cr₂Ti)C₂, (MO₂Ti)C₂, (W₂Ti)C₂,
Ti₄N₃, (Ti, Nb)₄C₃, (Ti₂Nb₂)C₃, (Ti₂Ta₂)C₃, (V₂Ti₂)C₃, (Cr₂Ti₂)C₃, (Mo₂Ti₂)C₃, (W₂Ti₂)C₃

Typically, in the above formula, M can be titanium, or titanium and vanadium, and X can be a carbon atom or a nitrogen atom. For example, a MAX phase is Ti₃AlC₂ and MXene is Ti₃C₂Tₛ (in other words, M is Ti, X is C, n is 2, and m is 3).

It is noted, in the present embodiment, MXene may contain remaining A atoms at a relatively small amount, for example, at 10% by mass or less with respect to the original amount of A atoms. The remaining amount of A atoms can be preferably 8% by mass or less, and more preferably 6% by mass or less. However, even if the remaining amount of A atoms exceeds 10% by mass, there may be no problem depending on the application and conditions of use of the electrode.

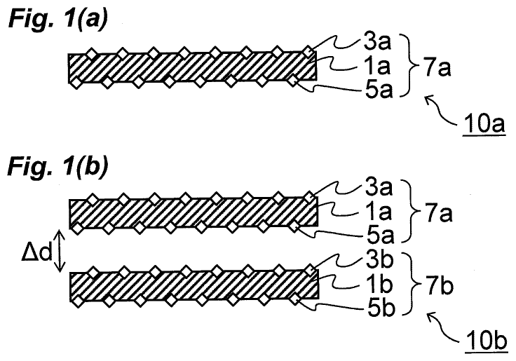

Hereinafter, MXene constituting layered material particles (MXene particles) will be described with reference to FIGS. 1(a) and 1(b). The metal cation-containing layered material particles (MXene particles containing a metal cation) according to the present embodiment have substantially the same skeleton as the layered material particles (MXene particles). In FIGS. 1(a) and 1(b), the structure corresponding to the skeleton of the MXene particles containing the metal cation is illustrated, and the metal cation is not shown in FIGS. 1(a) and 1(b).

The layered material particle (MXene particle) according to the present embodiment is an aggregate including a plurality of layers of a monolayered MXene 10a (single-layer MXene) schematically depicted in FIG. 1(a) (it is noted that, as described above, the metal cation is not shown). More specifically, the MXene 10a is a MXene layer 7a having a layer body represented by MₘXₙ (MₘXₙ layer) 1a and modifiers or terminals T3a, 5a existing on a surface of the layer body 1a (more specifically, on at least one of both surfaces facing opposite from each other, of each layer). Therefore, the MXene layer 7a is also represented by “MₘXₙTₛ”, wherein s is any number.

In the MXene particle according to the present embodiment, the MXene may be composed of one layer or a plurality of layers. Examples of the MXene composed of a plurality of layers (multilayer MXene) include, but are not limited to, a two-layered MXene 10b as schematically illustrated in FIGS. 1(b). 1b, 3b, 5b, and 7b in FIG. 1(b) are the same as 1a, 3a, 5a, and 7a in FIG. 1(a) described above. Two adjacent MXene layers (e.g., 7a and 7b) in the multilayer MXene may not necessarily be completely separated from each other, but may be partially in contact with each other. The MXene 10a is one that exists as a single layer resulting from separation of the multilayer MXene 10b into segments, and may exist as a mixture of the single-layer MXene 10a and the multilayer MXene 10b with some multilayer MXene 10b remaining unseparated. Even when the multilayer MXene is included, the multilayer MXene is preferably MXene having a small number of layers obtained through a delamination treatment. The “small number of layers” means, for example, that the number of stacked MXene layers is 10 or less. Hereinafter, the “multilayer MXene having a small number of layers” may be referred to as “few-layer MXene”. The thickness in the stacking direction of the few-layer MXene may be 15 nm or less, and may be 10 nm or less. The single-layer MXene and the few-layer MXene may be collectively referred to as “single-layer/few-layer MXene”.

Most of the MXene may be single-layer/few-layer MXene. When most of MXene is single-layer/few-layer MXene, the specific surface area of the MXene can be made larger than that of multilayer MXene. As a result, for example, when a laminate is used for applications requiring conductivity, deterioration of conductivity over time can be suppressed. For example, the single-layer/few-layer MXene in which the number of the stacked layers of MXene is 10 or less and the thickness is 15 nm or less, preferably 10 nm or less may account for, for example, 80% by volume or more, 90% by volume or more, or 95% by volume or more in the whole MXene. In addition, the volume of the single-layer MXene may be larger than the volume of the few-layer MXene. Since the true density of these MXenes does not greatly vary depending on the existence form, it can be said that the mass of the single-layer MXene is larger than the mass of the few-layer MXene. In the case of these relationships, the specific surface area of MXene can be increased, and deterioration of conductivity over time can be suppressed in use for the applications in which conductivity is required. For example, the film may be formed of only the single-layer MXene.

Although the present embodiment is not limited, the thickness of each layer of MXene, which corresponds to the MXene layers 7a and 7b, may be, for example, not less than 1 nm and not more than 30 μm, and may be, for example, not less than 1 nm and not more than 5 nm, or not less than 1 nm and not more than 3 nm, which can vary mainly depending on the number of M atom layers included in each layer. For individual laminates of the multilayer MXene that may be included, the inter-layer distance (or gap dimension, denoted as Δd in FIG. 1(b)) is, for example, not less than 0.8 nm and not more than 10 nm, particularly not less than 0.8 nm and not more than 5 nm, and more particularly about 1 nm, and the total number of layers may be not less than 2 and not more than 20,000.

The metal cation-containing layered material particle contains a metal cation as the name indicates. The type of the metal cation is not limited, but in view of easy insertion between layers of the layered material particles, a cation of an alkali metal is preferable, and it is more preferable to contain at least one cation among a Li cation, a Na cation, and a K cation. It is particularly preferable that the metal cation is at least one cation among a Li cation, a Na cation, and a K cation.

The content of the metal cation is 0.004 mol (0.004 mol/g) or more per gram of the film containing the metal cation-containing layered material particles (metal cation-containing MXene film, conductive film). It is considered that many metal cations are intercalated between layers of MXene. The presence of a certain amount or more of metal cations in the MXene film as a precursor film makes it possible to prevent diffusion of ions from a medium containing ions, for example, a conductive gel. Although the present disclosure is not bound by any theory, it is considered that the following effects are exerted by setting the content of the metal cation to a certain value or more. That is, it is considered that, heretofore, on performing discharge from a capacitor to an electrode, ions in a conductive gel in the electrode have been intercalated into MXene, deviation of ions has been caused between electrodes, and the inter-electrode potential has increased. However, it is considered that when the content of the metal cation is set to 0.004 mol/g or more and MXene is rich in ions in advance, further intercalation of ions due to discharge from the capacitor is suppressed, deviation of ions between electrodes is suppressed, so that an increase in inter-electrode potential can be suppressed. The content of the metal cation is preferably 0.006 mol or more per gram of the film containing the metal cation-containing layered material particles. The content of the metal cation is preferably as large as possible, but from the viewpoint of ease of manufacture and the like, it can be set to 0.1 mol or less (0.1 mol/g or less) per gram of the film containing the metal cation-containing layered material particles. The content of the metal cation may be 0.05 mol/g or less.

The electrode according to the present embodiment includes at least the film and a conductive gel portion. The electrode may be formed of only the conductive film and the conductive gel portion, or may include the conductive film, the conductive gel portion, and for example, a substrate.

The conductive gel portion in the electrode of the present embodiment can be formed of, for example, a gel material in which a solvent such as water or a humectant, a conductive material, or the like is held in a three-dimensional polymer matrix. As the gel material, for example, TECHNOGEL (registered trademark) of Sekisui Kasei Co., Ltd. can be adopted.

Examples of the electrode of the present embodiment include an electrode in a solid state and an electrode in a flexible and soft state.

When the electrode of the present embodiment has a substrate, the film and the substrate may be in direct contact with each other. The material of the substrate is not particularly limited. The substrate may be formed of a conductive material. Examples of the conductive material include at least one material among metal materials specified by gold, silver, copper, platinum, nickel, titanium, tin, iron, zinc, magnesium, aluminum, tungsten, and molybdenum, and a conductive polymer. The substrate may have, on a surface in contact with the conductive film according to the present embodiment, a film with conductivity, such as a metal film, different from the conductive film according to the present embodiment. Alternatively, the substrate may be formed of an organic material. Examples of the organic material include flexible organic materials, and examples thereof include a thermoplastic polyurethane elastomer (TPU), a PET film, and a polyimide film.

### Application of Electrode

The electrode of the present embodiment can be used for any suitable application. Although not particularly limited, examples thereof may include a biosignal sensing electrode, a capacitor electrode, a battery electrode, and a sensor electrode. Details of these applications will be described below.

The biosignal sensing electrode is an electrode for acquiring a biological signal. The biosignal sensing electrode may be, for example, but is not limited to, an electrode for measuring electroencephalogram (EEG), electrocardiogram (ECG), electromyogram (EMG), and electrical impedance tomography (EIT).

The capacitor may be an electrochemical capacitor. The electrochemical capacitor is a capacitor utilizing a capacitance developed due to a physicochemical reaction between an electrode (electrode active material) and ions in an electrolytic solution (electrolyte ions), and can be used as a device that stores electric energy (power storage device). The battery may be a repeatedly chargeable and dischargeable chemical battery. The battery may be, for example, but is not limited to, a Li ion battery, a magnesium ion battery, a lithium sulfur battery, or a Na ion battery.

The sensor electrode is an electrode for detecting a target substance, state, abnormality, or the like. The sensor may be, for example, but is not limited to, a gas sensor, a biosensor (i.e., a chemical sensor utilizing a molecular recognition mechanism of biological origin).

The electrode of the present embodiment is preferably used as a biosignal sensing electrode. As described above, it is considered that when the electrode including the film formed of MXene rich in metal cations and the conductive gel portion is used, for example, as a disposable electrocardiogram electrode as described above, a small deviation of ions between electrodes is caused when discharge from a capacitor is performed, and a low impedance is exhibited. As a result, it is considered that an improved sensitivity is exhibited when the electrode is used as a biosignal sensing electrode.

### Embodiment 3: Method for Manufacturing Electrode

A method for manufacturing an electrode according to the present embodiment will be described in detail, but the present disclosure is not limited to such an embodiment.

One method for manufacturing an electrode (a first manufacturing method) of the present embodiment is a method for manufacturing an electrode, the method comprising:
(a) preparing layered material particles each including one layer or a plurality of layers, the one layer or each of the plurality of layers including a layer body represented by the following formula:

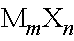

wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein T is at least one selected from the group consisting of a hydroxy group, a fluorine atom, a chlorine atom, an oxygen atom, and a hydrogen atom;
(b) mixing a dispersion liquid containing the layered material particles with an aqueous solution containing a metal cation to obtain a dispersion liquid containing metal cation-containing layered material particles;
(c) obtaining a film containing the metal cation-containing layered material particles, in which a content of the metal cation is 0.004 mol or more per gram of the film, using a dispersion liquid containing the metal cation-containing layered material particles; and
(d) forming a conductive gel portion on at least one surface of the film.

Another method for manufacturing an electrode (a second manufacturing method) of the present embodiment is a method for manufacturing an electrode, the method comprising:
(A) preparing layered material particles each including one layer or a plurality of layers, the one layer or each of the plurality of layers including a layer body represented by the following formula:

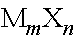

wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein T is at least one selected from the group consisting of a hydroxy group, a fluorine atom, a chlorine atom, an oxygen atom, and a hydrogen atom;
(B) obtaining a precursor film containing the layered material particles using a dispersion liquid containing the layered material particles;
(C) bringing the precursor film into contact with an aqueous solution containing a metal cation to obtain a film containing metal cation-containing layered material particles, in which a content of the metal cation is 0.004 mol or more per gram of the film; and
(D) forming a conductive gel portion on at least one surface of the film.

Hereinafter, each step of the first manufacturing method and the second manufacturing method will be described in detail. The step (a) and the step (A), and the step (d) and the step (D), which are common to these two manufacturing methods, will be collectively described.

### Step (a) and Step (A)

First, a prescribed precursor is prepared. The prescribed precursor that can be used in the present embodiment is a MAX phase that is a precursor to MXene, and is represented by a formula below:

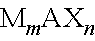

wherein M is at least one metal of Group 3, 4, 5, 6, or 7 and includes Ti,
X is a carbon atom, a nitrogen atom, or a combination thereof,
A is at least one element of Group 12, 13, 14, 15, or 16,
n is not less than 1 and not more than 4, and
m is more than n but not more than 5.

Said M, said X, said n, and said m are as described for MXene. A is at least one element of Group 12, 13, 14, 15, or 16, and is usually a Group A element, typically a Group IIIA element or a Group IVA element, and more specifically may comprise at least one element selected from the group consisting of Al, Ga, In, Ti, Si, Ge, Sn, Pb, P, As, S, and Cd, and is preferably Al.

The MAX phase has a crystal structure in which a layer constituted of A atoms is located between two layers each represented by MmXn (each layer can have a crystal lattice in which each X is located in an octahedral array of M). When typically m=n+1, but not limited thereto, the MAX phase includes repeating units in which each one layer of X atoms is disposed in between adjacent layers of n+1 layers of M atoms (these are also collectively referred to as an “MₘXₙ layer”), and a layer of A atoms (“A atom layer”) is disposed as a layer next to the (n+1)th layer of M atoms.

The MAX phase can be produced by a known method. For example, a TiC powder, a Ti powder, and an Al powder are mixed in a ball mill, and the resulting mixed powder is calcined under an Ar atmosphere to afford a calcined body (block-shaped MAX phase). Thereafter, the calcined body obtained is pulverized with an end mill to afford a powdered MAX phase for the next step.

The surface of the MₘXₙ layer exposed through the removal of the A atom layer (and, in some cases, also a part of M atoms) as a result of selective etching (removal and, in some cases, also layer separation) of A atoms (and, in some cases, also a part of M atoms) from the MAX phase is modified by hydroxy groups, fluorine atoms, chlorine atoms, oxygen atoms, hydrogen atoms, etc., existing in an etching liquid (usually, an aqueous solution of a fluorine-containing acid is used, but not limited thereto), so that the surface is terminated.

The etching can be carried out using an etching liquid containing F⁻, and a method using, for example, a mixed liquid of lithium fluoride and hydrochloric acid, a method using hydrofluoric acid, or the like may be used. The etching liquid contains a metal compound containing a monovalent metal ion, and an intercalation treatment of the monovalent metal ion may be performed simultaneously with the etching. Examples of the metal compound containing a monovalent metal ion include those to be used in the intercalation treatment described below. The content of the metal compound containing a monovalent metal ion in the etching liquid is preferably 0.001% by mass or more. The content is more preferably 0.01% by mass or more, and still more preferably 0.1% by mass or more. On the other hand, from the viewpoint of dispersibility in a solution, the content of the metal compound containing a monovalent metal ion in the etching liquid is preferably 10% by mass or less, and more preferably 1% by mass or less.

After the etching, the layer separation of MXene (delamination, that is, separating multilayer MXene into single-layer MXene) may be appropriately promoted by any suitable post-treatment (e.g., ultrasonic treatment, handshake, automatic shaker, or the like). Since the shear force of an ultrasonic treatment is excessively large so that the MXene can be destroyed, it is desirable to apply an appropriate shear force by handshake, an automatic shaker or the like, when it is desired to obtain a two-dimensional MXene (preferably single-layer MXene) having a larger aspect ratio.

For the layer separation of MXene, an intercalation treatment and delamination described below may be performed.

### Intercalation Treatment

For example, an intercalation treatment of monovalent metal ions including a step of mixing the etching product obtained by the etching treatment with a metal compound containing a monovalent metal ion may be performed. Examples of the monovalent metal ion constituting the metal compound containing a monovalent metal ion include alkali metal ions such as a Li ion, a Na ion, and a K ion, a copper ion, a silver ion, and a gold ion. Examples of the metal compound containing a monovalent metal ion include ionic compounds in which the metal cation is bonded to a cation. Examples thereof include an iodide, a phosphate, a sulfide salt including a sulfate, a nitrate, an acetate, and a carboxylate of the metal ion. A Li ion is preferable as the monovalent metal ion as described above, and metal compounds containing a Li ion are preferable as the metal compound containing a monovalent metal ion, ionic compounds of a Li ion are more preferable, and one or more among an iodide, a phosphate, and a sulfide salt of a Li ion are still more preferable. The use of a Li ion as a metal ion is considered to assist the formation of a monolayer due to the fact that water hydrated to the Li ion has the most negative dielectric constant.

The content of the metal compound containing a monovalent metal ion accounting for in the formulation for the intercalation treatment of a monovalent metal ion is preferably 0.001% by mass or more. The content is more preferably 0.01% by mass or more, and still more preferably 0.1% by mass or more. On the other hand, from the viewpoint of dispersibility in the solution, the content of the metal compound containing a monovalent metal ion is preferably 10% by mass or less, and more preferably 1% by mass or less.

### Delamination

Delamination may be performed using an intercalation product obtained by intercalation. For example, delamination includes a step of centrifuging the intercalation product, discarding the supernatant, and then washing the remaining precipitate with water. Conditions for the delamination treatment are not particularly limited. The dispersion medium to be used for delamination is not particularly limited, and the delamination may be performed using one or more of a polar organic dispersion medium and an aqueous dispersion medium. A process of adding one or more of the polar organic dispersion medium and the aqueous dispersion medium, stirring the mixture, centrifuging the mixture, and collecting the supernatant liquid is repeated once or more, preferably twice or more, and 10 times or less, whereby a supernatant containing a single-layer/few-layer MXene may be obtained as a delamination product. Alternatively, by centrifuging the supernatant, followed by discarding the supernatant resulting from the centrifugation, a clay containing a single-layer/few-layer MXene may be obtained as a delamination product.

### Step (b)

In the first manufacturing method, the dispersion liquid containing the layered material particles is mixed with an aqueous solution containing a metal cation to afford a dispersion liquid containing metal cation-containing layered material particles.

The dispersion liquid containing layered material particles can be obtained, for example, as a slurry containing the layered material particles by stirring and mixing the single-layer/few-layer MXene-containing clay obtained by the delamination with an aqueous dispersion medium such as pure water.

The type of the metal cation contained in the aqueous solution containing the metal cation is not limited, but in view of easy insertion between layers of the layered material particles and the like, a cation of an alkali metal is preferable, and it is more preferable that the solution contains at least one cation among a Li cation, a Na cation, and a K cation. It is particularly preferable that the metal cation is at least one cation among a Li cation, a Na cation, and a K cation. The concentration of the metal cation in the aqueous solution containing the metal cation is not particularly limited as long as the amount of the metal cation per gram of the finally obtained film is 0.004 mol or more. From the viewpoint of easily intercalating metal cations between layers of the layered material particles by mixing with the dispersion liquid containing the layered material particles, it is preferable to use an aqueous solution containing 10% by mass to 30% by mass of, for example, a chloride, a fluoride, a bromide, an iodide, a sulfate, a nitrate, or a phosphate of a metal cation.

The method for mixing the dispersion liquid containing the layered material particles with the aqueous solution containing the metal cation is not particularly limited, and stirring may be performed by a known method. The liquid temperature at the time of the mixing is not particularly limited, and may be room temperature.

### Step (c)

In the first manufacturing method, a film containing metal cation-containing layered material particles is obtained using the dispersion liquid containing metal cation-containing layered material particles. For formation of the film, a dispersion liquid of metal cation-containing MXene such as a metal cation-containing MXene slurry prepared by diluting the clay containing metal cation-containing MXene with a medium liquid can be used. The dispersion liquid may be a suspension. The method for forming the film using the dispersion liquid of a metal cation-containing MXene is not particularly limited. The dispersion liquid of a metal cation-containing MXene may be applied to the substrate as received or after being appropriately adjusted (for example, dilution with a medium liquid, or addition of a binder). Examples of the application method include a method of performing spray application using a nozzle such as a one-fluid nozzle, a two-fluid nozzle, or an air brush (a method using a spray coater), a method such as slit coating, screen printing, or metal mask printing using a table coater, a comma coater, or a bar coater, spin coating, dip coating, and dropping. Examples of the medium liquid include an aqueous medium liquid and an organic medium liquid. The medium liquid that constitutes the dispersion liquid of a metal cation-containing MXene is typically water, and in some cases, other liquid substances may be contained in a relatively small amount (e.g., 30% by mass or less, preferably 20% by mass or less based on the whole mass) in addition to water. Examples of the organic medium liquid include N-methylpyrrolidone, N-methylformamide, N,N-dimethylformamide, ethanol, methanol, dimethylsulfoxide, ethylene glycol, and acetic acid.

When the film is formed using a spray coater, a film (an electrode) before drying can be formed by applying, for example, a metal cation-containing MXene slurry to a substrate such as PET or polyimide with the atomization pressure to not less than 0.1 MPa and not more than 0.5 MPa, the distance between the nozzle tip and the substrate set to not less than 10 cm and not more than 25 cm, the liquid feeding amount to not less than 0.1 mL/s and not more than 10 mL/s, the sweep rate to not less than 1 mm/s and not more than 30 mm/s, and the stage heater to not less than 30° C. to not more than 60° C., once or multiple times.

Besides the preparation of a film by spraying as described above, a film may be prepared by subjecting a metal cation-containing MXene slurry to suction filtration. More specifically, the concentration of the metal cation-containing MXene slurry is appropriately adjusted (dilute, for example, with an aqueous medium liquid, as necessary), and the slurry is then subjected to suction filtration through a filter (this filter may be one that constitutes a prescribed member together with the metal cation-containing MXene film, or may be finally separated from the metal cation-containing MXene film) installed in a Nutsche or the like, thereby at least partially removing the aqueous medium liquid, whereby a film can be formed on the filter. The filter is not particularly limited, a membrane filter or the like can be used.

The substrate may or may not be included. When the substrate is included, the material constituting the substrate is not particularly limited, and the substrate may be formed of any appropriate material. The substrate may be, for example, a resin film, a metal foil, a printed wiring board, a mounted electronic component, a metal pin, a metal wiring, a metal wire, or the like. For example, a substrate formed of a metal material, resin, or the like suitable for a biosignal sensing electrode can be appropriately adopted. By applying the metal cation-containing MXene slurry to any appropriate substrate (this substrate may be one that constitutes a prescribed member together with a metal cation-containing MXene film, or may be finally separated from a metal cation-containing MXene film), a metal cation-containing MXene film can be formed on the substrate.

Drying may be performed under mild conditions such as natural drying (typically, the item to be dried is disposed in an air atmosphere at normal temperature and normal pressure) or air drying (blowing air), or may be performed under relatively active conditions such as hot air drying (blowing heated air), heat drying, and/or vacuum drying. In the present embodiment, “drying” means removing the medium liquid that may be present in the film. Drying may be performed, for example, at a temperature of 400° C. or less using a normal pressure oven or a vacuum oven. For example, drying may be performed in the ranges of not less than 30° C. and not more than 200° C. and not less than 30 minutes and not more than 24 hours.

The formation and drying of the metal cation-containing MXene film may be appropriately repeated until a desired film thickness is obtained. For example, a combination of spraying and drying may be repeated multiple times. In the metal cation-containing MXene film, a liquid component derived from the liquid medium of the slurry may remain or may be substantially absent.

### Step (B)

In the second manufacturing method, a precursor film containing layered material particles is obtained using the dispersion liquid containing the layered material particles. For formation of the precursor film, a dispersion liquid of layered material particles (MXene particles), such as a MXene slurry prepared by diluting the single-layer/few-layer MXene-containing clay with a medium liquid can be used. The dispersion liquid may be a suspension. The method for forming a precursor film using a dispersion liquid of MXene particles is not particularly limited. The dispersion liquid of MXene particles may be applied to a substrate as received or after being appropriately adjusted (for example, dilution with a medium liquid, or addition of a binder). Examples of the application method include a method of performing spray application using a nozzle such as a one-fluid nozzle, a two-fluid nozzle, or an air brush (a method using a spray coater), a method such as slit coating, screen printing, or metal mask printing using a table coater, a comma coater, or a bar coater, spin coating, dip coating, and dropping. Examples of the medium liquid include an aqueous medium liquid and an organic medium liquid. The medium liquid that constitutes the dispersion of MXene particles is typically water, and in some cases, other liquid substances may be contained in a relatively small amount (e.g., 30% by mass or less, preferably 20% by mass or less based on the whole mass) in addition to water. Examples of the organic medium liquid include N-methylpyrrolidone, N-methylformamide, N,N-dimethylformamide, ethanol, methanol, dimethylsulfoxide, ethylene glycol, and acetic acid.

When the film is formed using a spray coater, a film before drying can be formed by applying, for example, a MXene slurry to a substrate such as PET or polyimide with the atomization pressure to not less than 0.1 MPa and not more than 0.5 MPa, the distance between the nozzle tip and the substrate set to not less than 10 cm and not more than 25 cm, the liquid feeding amount to not less than 0.1 mL/s and not more than 10 mL/s, the sweep rate to not less than 1 mm/s and not more than 30 mm/s, and the stage heater to not less than 30° C. to not more than 60° C., once or multiple times.

Besides the preparation of a precursor film by spraying as described above, a precursor film may be prepared by subjecting the slurry or the supernatant containing MXene particles obtained by the delamination to suction filtration. More specifically, for example, the supernatant containing MXene particles as a dispersion of MXene particles is appropriately adjusted (for example, diluted with an aqueous medium liquid), and then subjected to suction filtration through a filter (this filter may be one that constitutes a prescribed member together with a precursor film, or may be finally separated from the precursor film) installed in a Nutsche or the like, thereby at least partially removing the aqueous medium liquid, whereby a precursor film can be formed on the filter. The filter is not particularly limited, a membrane filter or the like can be used. By performing the suction filtration, a precursor film can be prepared without using the binder or the like. When the MXene particles of the present embodiment are used, a precursor film can be prepared without using a binder or the like in this manner.

The substrate may or may not be included. When the substrate is included, the material constituting the substrate is not particularly limited, and the substrate may be formed of any appropriate material. The substrate may be, for example, a resin film, a metal foil, a printed wiring board, a mounted electronic component, a metal pin, a metal wiring, a metal wire, or the like. For example, a substrate formed of a metal material, resin, or the like suitable for a biosignal sensing electrode can be appropriately adopted. By applying the dispersion of MXene to any appropriate substrate (this substrate may be one that constitutes a prescribed member together with a precursor film, or may be finally separated from a precursor film), a precursor film can be formed on the substrate.

Drying may be performed under mild conditions such as natural drying (typically, the item to be dried is disposed in an air atmosphere at normal temperature and normal pressure) or air drying (blowing air), or may be performed under relatively active conditions such as hot air drying (blowing heated air), heat drying, and/or vacuum drying. In the present embodiment, “drying” means removing the medium liquid that may be present in the precursor film. Drying may be performed, for example, at a temperature of 400° C. or less using a normal pressure oven or a vacuum oven. For example, drying may be performed in the ranges of not less than 30° C. and not more than 200° C. and not less than 30 minutes and not more than 24 hours.

The formation and drying of the precursor film may be appropriately repeated until a desired thickness of the precursor film is obtained. For example, a combination of spraying and drying may be repeated multiple times. By performing the suction filtration, a precursor film can be formed without including a binder. In the precursor film, a liquid component derived from the liquid medium of the slurry may remain or may be substantially absent.

### Step (C)

In the second manufacturing method, a film containing metal cation-containing layered material particles (metal cation-containing MXene film) is obtained by bringing the precursor film into contact with an aqueous solution containing a metal cation.

The type of the metal cation contained in the aqueous solution containing the metal cation is not limited, but in view of easy insertion between layers of the layered material particles and the like, a cation of an alkali metal is preferable, and it is more preferable that the solution contains at least one cation among a Li cation, a Na cation, and a K cation. It is particularly preferable that the metal cation is at least one cation among a Li cation, a Na cation, and a K cation. The concentration of the metal cation in the aqueous solution containing the metal cation is not particularly limited as long as the amount of the metal cation per gram of the finally obtained film is 0.004 mol or more. From the viewpoint of easily intercalating metal cations between layers of the layered material particles by bringing into contact with the precursor film, it is preferable to use an aqueous solution containing, for example, a chloride, a fluoride, a bromide, an iodide, a sulfate, a nitrate, or a phosphate of a metal cation in an amount being 50 to 95% by mass of the saturated dissolution amount at 25° C.

The temperature of the aqueous solution containing a metal cation when brought into contact with the precursor film may be room temperature (normal temperature). The method for bringing the precursor film into contact with the aqueous solution containing a metal cation is not particularly limited, and examples thereof include a method in which the precursor film is immersed in the aqueous solution containing a metal cation, and a method in which spray coating is performed using a nozzle in order to bring the aqueous solution into contact with the entire or a part of at least one surface of the precursor film. The contact time (immersion time in the case of immersion) can be set to, for example, 30 minutes to 24 hours.

### Step (d) and Step (D)

A conductive gel portion is formed on at least one surface of the obtained metal cation-containing MXene film. As the conductive gel, a conductive gel contained in the above-described electrode can be employed. The conductive gel is formed on at least one surface of the metal cation-containing MXene film by coating, bonding, or the like. The place where the conductive gel portion is formed on at least one surface of the film may be the entire surface or a part of the surface, as necessary.

Although the electrode in one embodiment of the present disclosure has been described in detail above, the present disclosure allows various modifications. It should be noted that the electrode of the present disclosure may be manufactured by a method different from the manufacturing methods in the embodiments described above.

### EXAMPLES

In the following, the present disclosure will be described more specifically with reference to Examples. The present disclosure is not limited by the following Examples, and can be implemented with appropriate modifications as long as the modifications can be consistent with the above-described and later-described gist, and all of them are included in the technical scope of the present disclosure.

### Examples 1 to 6: First Manufacturing Method, Comparative Examples 2 to 4

### 1. Preparation of Layered Material Particles (MXene)

(1) Preparation of a precursor (MAX), (2) etching of the precursor, (3) washing after the etching, (4) Li intercalation, and (5) delamination each described in detail below were performed in order, affording MXene particles first.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined in an Ar atmosphere at 1350° C. for 2 hours. The calcined body (block-shaped MAX) thus obtained was pulverized with an end mill to a maximum size of 40 μm or less. Thereby, Ti₃AlC₂ particles were obtained as a precursor (powdered MAX).

### (2) Etching of Precursor (MAX)

Using the Ti₃AlC₂ particles (powder) prepared by the above method, etching was performed under the following etching conditions, affording a solid-liquid mixture (slurry) containing a solid component derived from the Ti₃AlC₂ powder.

### Etching Conditions

Precursor: Ti₃AlC₂ (sieved with a mesh size of 45 μm)
Etching liquid composition: 49% HF 6 mL, H₂O 18 mL HCl (12M) 36 mL
Amount of precursor input: 3.0 g
Etching container: 100 mL Aiboy
Etching temperature: 35° C.
Etching time: 24 h
Stirrer rotation speed: 400 rpm

### (3) Washing After Etching

The slurry was equally divided into two portions and inserted into two 50 mL centrifuge tubes. Thereafter, the mixture was centrifuged at 3500 G for 5 minutes using a centrifuge, and then the supernatant was discarded. Thereafter, (i) 35 mL of pure water was added to the remaining precipitate in each centrifuge tube, (ii) stirring was performed by handshake, (iii) centrifugation was performed at 3500 G for 5 minutes, and (iv) the supernatant was removed. The steps (i) to (iv) were repeated 10 times. Finally, centrifugation was performed at 3500 G for 5 minutes to obtain a Ti₃C₂Tₛ-water medium clay.

### (4) Li Intercalation

The Ti₃C₂Tₛ-water medium clay prepared by the above method was stirred at not less than 20° C. and not more than 25° C. for 12 hours using LiCl as a Li-containing compound in accordance with the following conditions of Li intercalation, whereby Li intercalation was performed. The detailed conditions of the Li intercalation are as follows.

### Conditions of Li Intercalation

Ti₃C₂Tₛ-water medium clay (MXene after washing): Solid content: 0.75 g
LiCl: 0.75 g
Intercalation container: 100 mL Aiboy
Temperature: not less than 20° C. and not more than 25° C. (room temperature)
Time: 12 h
Stirrer rotation speed: 800 rpm

### (5) Delamination and Washing With Water

The slurry obtained by Li intercalation was charged into a 50 mL centrifuge tube, centrifuged under the condition of 3500 G using a centrifuge, and then the supernatant was discarded. Next, (i) 40 mL of pure water was added to the remaining precipitate, and the mixture was stirred for 15 minutes with a shaker, then (ii) centrifuged at 3500 G, and (iii) the supernatant was collected as a single-layer/few-layer MXene-containing liquid. The operations (i) to (iii) were repeated 4 times in total, affording a single-layer/few-layer MXene-containing supernatant. Further, this supernatant was centrifuged under the conditions of 4300 G and 2 hours using a centrifuge, and then the supernatant was discarded, affording a single-layer/few-layer MXene-containing MXene clay as a remaining precipitate.

### 2. Preparation of Dispersion Liquid Containing MXene

A prescribed amount of the MXene clay obtained in the above 1. was taken into a 50 mL centrifuge tube, and pure water was added thereto such that the concentration of MXene was 1.5% by mass. Subsequently, the mixture was stirred with a shaker for 15 minutes to form a dispersion liquid containing layered material particles, affording a slurry containing the layered material particles.

### 3. Preparation of Dispersion Liquid Containing Metal Cation-Containing Layered Material Particles

The slurry containing the layered material particles, a 15% by mass aqueous LiCl solution, a 15% by mass aqueous NaCl solution, or a 15% by mass aqueous KCl solution prepared in advance, and pure water were mixed, affording an aqueous solution containing metal cation-containing layered material particles containing 1.5% by mass of MXene and each metal cation having a content shown in Table 1.

### 4. Preparation of Film Containing Metal Cation-Containing Layered Material Particles

The dispersion liquid (slurry) containing the metal cation-containing layered material particles obtained in the above 3. was placed in a 25 ml syringe. The syringe was set in a spray coater. Next, the atomization pressure in the spray coater was set to 0.5 MPa, the distance between the tip of the nozzle and the substrate was set to 15 cm, the liquid feeding amount was set to 5 mL/s, the sweep rate was set to 150 mm/s, and the stage heater was set to 45° C. Then, the dispersion liquid was applied to a substrate (polyimide film) 15 times using the spray coater. Subsequently, drying was performed at 80° C. for 2 hours using a normal pressure oven, affording a film containing metal cation-containing layered material particles.

### Examples 7 to 9: Second Manufacturing Method

### 1. Preparation of Layered Material Particles (MXene)

Layered material particles were prepared in the same manner as in Examples 1 to 6.

### 2. Preparation of Dispersion Liquid Containing MXene

A prescribed amount of the MXene clay obtained in the above 1. was taken into a 50 mL centrifuge tube, and pure water was added thereto such that the concentration of MXene was 1.5% by mass. Subsequently, the mixture was stirred with a shaker for 15 minutes to form a dispersion liquid containing layered material particles, affording a slurry containing the layered material particles.

### 3. Preparation of Precursor Film (Film Containing Layered Material Particles)

The dispersion liquid (slurry) containing MXene obtained in the above 2. was placed in a 25 ml syringe. The syringe was set in a spray coater. Next, the atomization pressure in the spray coater was set to 0.5 MPa, the distance between the tip of the nozzle and the substrate was set to 15 cm, the liquid feeding amount was set to 5 mL/s, the sweep rate was set to 150 mm/s, and the stage heater was set to 45° C. Then, the dispersion liquid was applied to a substrate (polyimide film) 15 times using the spray coater. Then, drying was performed at 80° C. for 2 hours using a normal pressure oven, affording a precursor film (film containing layered material particles).

### 4. Preparation of Film Containing Metal Cation-Containing Layered Material Particles

First, an aqueous LiCl solution, an aqueous NaCl solution, and an aqueous KCl solution each having a concentration of 90% by mass of the saturated dissolution amount at 25° C. were prepared. The precursor film prepared in the above 3. was immersed in each of the aqueous solutions for 2 hours, and then taken out. Thereafter, moisture was wiped off, and the resulting film was dried in air for 1 hour, affording a film (film thickness: about 2 μm) containing metal cation-containing layered material particles.

As Comparative Example 1, a film containing layered material particles containing no metal cation (corresponding to a sole MXene film or a precursor film) was also prepared.

### Evaluation

### Measurement of Content of Metal Cation

The content of metal cations contained in the film was determined by collecting the film in a container, adding an acid (dilute nitric acid, dilute sulfuric acid, and hydrofluoric acid) thereto, and dissolving the metal in a microwave sample digester UltraWAVE ECR manufactured by Milestone General K. K. to prepare an aqueous solution, then measuring the content of each metal in the aqueous solution by an inductively coupled plasma (ICP) emission spectrometer iCAP7400 radial manufactured by Thermo Fisher Scientific K. K.

### Implementation of EC12 Test 4

### 1. Preparation of Film

The films containing metal cation-containing layered material particles prepared as described above (Examples 1 to 9, Comparative Examples 2 to 4) and the sole MXene film (Comparative Example 1) were each cut into 6 sheets having a size of 2 cm×2.5 cm per level.

### 2. Preparation of Electrode

CR Grade TECHNOGEL (registered trademark, sheet-shaped) manufactured by Sekisui Kasei Co., Ltd. was cut into a 2 cm square, and one sheet of CR Grade TECHNOGEL was attached to one sheet of the film cut out in the above 1., affording an electrode having a gel portion.

### 3. Implementation of Test

The test described in 4.2.2.4 of ANSI/AAMI EC12:2000/(R)2020 (hereinafter, this test is abbreviated as Test 4) was implemented by the following method.
(1) Two electrodes having a gel portion were prepared, bonded together on their gel portions, and then connected to a measurement device Surface Electrode Analysis Meter manufactured by QC Integrated Solutions Inc. using a separately provided instrument.
(2) The capacitor incorporated in the measurement device was charged to 200 V, and then the charged electricity was discharged to the set of the two electrodes bonded together on their gel portions.
(3) After 5 seconds, 15 seconds, 25 seconds, and 35 seconds from the end of discharge of the capacitor charged to 200 V, the inter-electrode potential was measured.
(4) The above (2) and (3) were performed consecutively 4 times in total.

### 4. Evaluation

When in all of the consecutive four measurements, the inter-electrode potential after 5 seconds was 100 mV or less, the rate of change in the inter-electrode potential during each 10-seconds period of from 5 seconds to 15 seconds, from 15 seconds to 25 seconds, and from 25 seconds to 35 seconds satisfied±10 mV or less, and the impedance at 10 Hz measured after the consecutive four measurements was 3 kΩ or less, it was evaluated that Test 4 was overcome (indicated as “G” in Table 1). On the other hand, when at least one of the above evaluation criteria was not satisfied, it was evaluated that Test 4 was not overcome (indicated as “NG” in Table 1).

TABLE 1

| | | Metal cation | | |
|---|---|---|---|---|
| | | | Content | Result of |
| | Preparation method | Type | (mol/g)* | Test 4 |
| Example 1 | First manufacturing | Li | 0.004 | G |
| | method | | | |
| Example 2 | First manufacturing | Li | 0.008 | G |
| | method | | | |
| Example 3 | First manufacturing | Na | 0.004 | G |
| | method | | | |
| Example 4 | First manufacturing | Na | 0.008 | G |
| | method | | | |
| Example 5 | First manufacturing | K | 0.004 | G |
| | method | | | |
| Example 6 | First manufacturing | K | 0.008 | G |
| | method | | | |
| Example 7 | Second manufacturing | Li | 0.009 | G |
| | method | | | |
| Example 8 | Second manufacturing | Na | 0.009 | G |
| | method | | | |
| Example 9 | Second manufacturing | K | 0.009 | G |
| | method | | | |
| Comparative | — (Sole MXene) | | | NG |
| Example 1 | | | | |
| Comparative | Same as the first | Li | 0.003 | NG |
| Example 2 | manufacturing method | | | |
| | except for the content | | | |
| | of metal cation | | | |
| Comparative | Same as the first | Na | 0.003 | NG |
| Example 3 | manufacturing method | | | |
| | except for the content | | | |
| | of metal cation | | | |
| Comparative | Same as the first | K | 0.003 | NG |
| Example 4 | manufacturing method | | | |
| | except for the content | | | |
| | of metal cation | | | |

*Molar amount of metal cation per 1 g of film

The electrodes of Examples 1 to 9, into which a certain amount or more of metal cations were introduced, overcame the test of 4.2.2.4 of ANSI/AAMI EC12:2000/(R)2020, which is a standard for disposable electrocardiogram electrodes. It is considered that by introducing metal cations in advance, when a voltage was applied in the test, further charging was minimized, and an increase in inter-electrode potential was minimized. In addition, since the increase in inter-electrode potential was suppressed, the discharge rate was reduced also during discharge, and the test of 4.2.2.4 of ANSI/AAMI EC12:2000/(R)2020 could be overcome. On the other hand, Comparative Example 1, in which no metal cation was introduced, and Comparative Examples 2 to 4, in which the introduction amount of metal cations was insufficient, failed in the test. In these Comparative Examples, it is considered that when the capacitor charged to 200 V discharged to the electrode, ions in the gel were intercalated into MXene, and ions were deviated between the electrodes, so that Test 4 could not be overcome.

The electrode according to the present disclosure can be utilized for any suitable application, and can be suitably used as a biosignal sensing electrode, but is not limited thereto.

### REFERENCE SIGNS LIST

1a, 1b Layer body (MₘXₙ layer)
3a, 5a, 3b, 5b Modifier or terminal T
7a, 7b MXene layer
10, 10a, 10b Layered material particles

1. An electrode comprising:
a film containing metal cation-containing layered material particles, wherein the metal cation-containing layered material particles each have one layer or a plurality of layers and a metal cation, the one layer or each of the plurality of layers includes a layer body represented by: 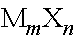
wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein Tis at least one selected from a hydroxy group, a fluorine atom, a chlorine atom, an oxygen atom, and a hydrogen atom, and a content of the metal cation is 0.004 mol or more per gram of the film; and
a conductive gel portion in contact with the film.

2. The electrode according to claim 1, wherein the metal cation includes at least one cation among a Li cation, a Na cation, and a K cation.

3. The electrode according to claim 1, wherein the metal cation is a cation of an alkali metal.

4. The electrode according to claim 1, wherein the electrode is configured as a biosignal sensing electrode.

5. The electrode according to claim 1, wherein the content of the metal cation is 0.004 mol per gram to 0.1 mol per gram of the film.

6. The electrode according to claim 1, wherein the content of the metal cation is 0.006 mol or more per gram of the film.

7. The electrode according to claim 1, wherein the content of the metal cation is 0.006 mol per gram to 0.05 mol per gram of the film.

8. A method for manufacturing an electrode, the method comprising:
(a) preparing layered material particles each including one layer or a plurality of layers, the one layer or each of the plurality of layers including a layer body represented by: 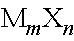
wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein T is at least one selected from a hydroxy group, a fluorine atom, a chlorine atom, an oxygen atom, and a hydrogen atom;
(b) mixing a dispersion liquid containing the layered material particles with an aqueous solution containing a metal cation to obtain a dispersion liquid containing metal cation-containing layered material particles;
(c) obtaining a film containing the metal cation-containing layered material particles, in which a content of the metal cation is 0.004 mol or more per gram of the film, using the dispersion liquid containing the metal cation-containing layered material particles; and
(d) forming a conductive gel portion on at least one surface of the film.

9. The method according to claim 8, wherein the metal cation includes at least one cation among a Li cation, a Na cation, and a K cation.

10. The method according to claim 8, wherein the metal cation is a cation of an alkali metal.

11. The method according to claim 8, wherein the content of the metal cation is 0.004 mol per gram to 0.1 mol per gram of the film.

12. The method according to claim 8, wherein the content of the metal cation is 0.006 mol or more per gram of the film.

13. The method according to claim 8, wherein the content of the metal cation is 0.006 mol per gram to 0.05 mol per gram of the film.

14. A method for manufacturing an electrode, the method comprising:
(A) preparing layered material particles each including one layer or a plurality of layers, the one layer or each of the plurality of layers including a layer body represented by: 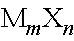
wherein M is at least one metal of Group 3, 4, 5, 6, or 7 and includes at least a Ti atom,
X is a carbon atom, a nitrogen atom, or a combination thereof,
n is not less than 1 and not more than 4, and
m is more than n but not more than 5; and
a modifier or terminal T existing on a surface of the layer body, wherein T is at least one selected from a hydroxy group, a fluorine atom, a chlorine atom, an oxygen atom, and a hydrogen atom;
(B) obtaining a precursor film containing the layered material particles using a dispersion liquid containing the layered material particles;
(C) bringing the precursor film into contact with an aqueous solution containing a metal cation to obtain a film containing metal cation-containing layered material particles, in which a content of the metal cation is 0.004 mol or more per gram of the film; and
(D) forming a conductive gel portion on at least one surface of the film.

15. The method according to claim 14, wherein a time for which the precursor film is kept in contact with the aqueous solution containing the metal cation is 30 minutes to 24 hours.

16. The method according to claim 14, wherein the metal cation includes at least one cation among a Li cation, a Na cation, and a K cation.

17. The method according to claim 14, wherein the metal cation is a cation of an alkali metal.

18. The method according to claim 14, wherein the content of the metal cation is 0.004 mol per gram to 0.1 mol per gram of the film.

19. The method according to claim 14, wherein the content of the metal cation is 0.006 mol or more per gram of the film.

20. The method according to claim 14, wherein the content of the metal cation is 0.006 mol per gram to 0.05 mol per gram of the film.
